(19) United States
(12) Patent Application Publication
(10) Pub. No.: US 2026/0201073 A1
(43) Pub. Date: Jul. 16, 2026

(54) IONIC LIQUID FOR CELLULOSE PROCESSING AND METHODS OF USE AND RECYCLING THEREOF

(71) Applicant: Chung Hwa Pulp Corporation, Taipei (TW)

(72) Inventors: HUI-WEN HSU, Taipei (TW); CHEN-HSIU HUNG, Taipei (TW); CHIA-HAO LIU, Taipei (TW); TAI-YUAN CHEN, Taipei (TW)

(21) Appl. No.: 19/445,864

(22) Filed: Jan. 12, 2026

Related U.S. Application Data
(60) Provisional application No. 63/744,895, filed on Jan. 14, 2025.

(51) Int. Cl. C08B 1/00; C07D 233/58; C07F 9/11

(52) U.S. Cl. CPC C08B 1/003; C07D 233/58; C07F 9/11

(57) ABSTRACT

An ionic liquid includes a cation and an anion, in which the cation has a structure represented by Formula (1) and the anion has a structure represented by Formula (2) as follows:

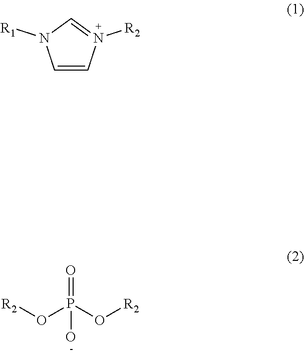

where R1 and R2 are defined as follows: (i) R1 is an isopropyl group and R2 is a methyl group or an ethyl group; or (ii) R1 is a propyl group and R2 is an ethyl group. A method for producing the ionic liquid and a method for spinning cellulose and recycling the ionic liquid are also provided. Furthermore, a cellulose-ionic liquid solution and a method for dissolving cellulose are provided as well.

### RELATED APPLICATIONS

This application claims priority to Provisional Application Ser. No. 63/744,895, filed on Jan. 14, 2025, the entire disclosure of which is incorporated herein by reference.

### BACKGROUND

The textile industry is highly resource-intensive and remains a significant contributor to global greenhouse gas emissions and solid waste generation. Currently, over one-third of textile fibers are derived from fossil-based sources. With global demand for textiles continuing to grow, there is an urgent need for innovative and sustainable strategies. However, the practical use of cellulose is hindered by its inherent insolubility in water and most common organic solvents, due to the extensive network of intra- and intermolecular hydrogen bonds (Chen et al., 2015; Pinkert et al., 2010). This presents a fundamental challenge in cellulose processing and limits its broader application. Therefore, the development of efficient, environmentally benign dissolution systems is not only a long-term research goal but a critical step toward enabling cellulose to serve as a viable, sustainable alternative to petroleum-derived materials.

Various solvents capable of directly dissolving cellulose have been identified, including LiCl/N,N-dimethylacetamide (DMAc) (McCormick et al., 1985), NaOH aqueous solution (Budtova & Navard, 2015), N-methylmorpholine N-oxide (NMMO) monohydrate (Fink et al., 2001), ionic liquids (ILs) (Swatloski et al., 2002), and deep eutectic solvents (DESs) (Freitas et al., 2024). Among these solvents, only NMMO monohydrate is employed on an industrial scale as a direct cellulose solvent, particularly in the textile fiber spinning process. Unfortunately, the presence of metal ions can lead to the decomposition and explosion of NMMO, necessitating substantial efforts to enhance the corresponding post-treatment technologies (Stepan et al., 2016).

Both ILs and DESs exhibit negligible vapor pressures, making them attractive for processes where minimizing solvent loss is critical (Alsoy Altinkaya, 2024). Their physicochemical properties can be finely tuned by modifying their constituent components, such as the hydrogen bond acceptors and donors in DESs, or the cation and anion combinations in ILs, to meet the requirements of specific applications (Verdia Barbará et al., 2023). However, in the context of cellulose processing, DESs face significant challenges related to solvent recovery and reuse, including decomposition and technical difficulties during recycling. These issues represent a major techno-economic and environmental limitation to the broader adoption of DES-based systems (Mariño et al., 2024).

### BRIEF DESCRIPTION OF THE DRAWINGS

Aspects of the present disclosure are best understood from the following detailed description when read with the accompanying FIGs. It is noted that, in accordance with the standard practice in the industry, various features are not drawn to scale. In fact, the dimensions of the various features may be arbitrarily increased or reduced for clarity of discussion.

FIG. 1 is a flowchart illustrating a method for preparing an ionic liquid according to some embodiments of the present disclosure.

FIG. 2 is a flowchart illustrating a method for spinning cellulose and recovering an ionic liquid according to some embodiments of the present invention.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. As used herein, the term “and/or” includes any and all combinations of one or more of the associated listed items. It will be appreciated that although these methods each illustrate a number of operations, steps, acts and/or features, not all of these operations, steps, acts and/or features are necessarily required, and other un-illustrated operations, acts and/or features may also be present. Also, the ordering of the operations, steps and/or acts in some embodiments can vary from what is illustrated in these figures. While in other implementations, some of the illustrated operations, steps and/or acts can be carried out concurrently with one another.

According to one aspect of the present disclosure, an ionic liquid is provided. The ionic liquid includes a cation and an anion, in which the cation has a structure represented by Formula (1) and the anion has a structure represented by Formula (2) as follows:

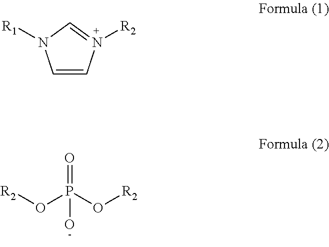

where R1 and R2 are defined as follows: (i) R1 is an isopropyl group and R2 is a methyl group or an ethyl group; or (ii) R1 is a propyl group and R2 is an ethyl group.

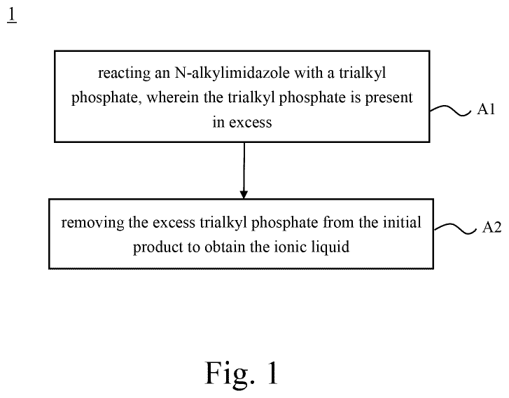

According to another aspect of the present disclosure, a method for producing an ionic liquid described hereinbefore in connection with Formula (1) and Formula (2) is provided. FIG. 1 is a flowchart illustrating a method 1 for producing an ionic liquid according to some embodiments of the present disclosure. The method 1 includes operation A1 and operation A2.

In operation A1, an N-alkylimidazole is reacted with a trialkyl phosphate, in which the trialkyl phosphate is present in excess, such that the N-alkylimidazole is substantially completely reacted, thereby obtaining an initial product. The reaction scheme of operation A1 is represented by Formula (3) as follows:

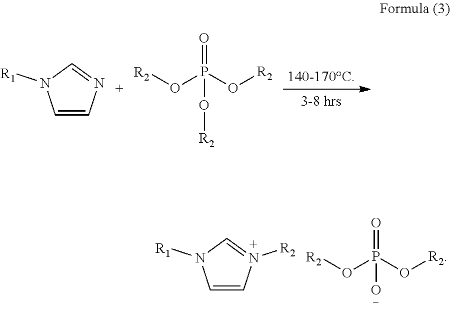

In operation A1, a molar ratio of the trialkyl phosphate to the N-alkylimidazole (trialkyl phosphate/N-alkylimidazole) is about 1.05 to about 1.15 according to some embodiments. In examples, the molar ratio of the trialkyl phosphate to the N-alkylimidazole may be about 1.05, about 1.10, or about 1.15. The use of an excess amount of the trialkyl phosphate ensures that the N-alkylimidazole is substantially completely reacted. In some embodiments, operation A1 includes reacting the N-alkylimidazole with the trialkyl phosphate at a temperature ranging from about 130° C. to about 170° C. for a time period of about 3 hours to about 8 hours to obtain the initial product. The reaction temperature may be, for example, about 130° C., about 140° C., about 150° C., about 160° C., or about 170° C. The reaction time period may be about 3 hours, about 4 hours, about 5 hours, about 6 hours, or about 8 hours.

In operation A2, the excess trialkyl phosphate is removed from the initial product to obtain the ionic liquid. As described in operation A1, the trialkyl phosphate is present in excess, and therefore operation A2 aims to remove the remaining (or unreacted) trialkyl phosphate from the initial product to obtain a purified ionic liquid.

In some embodiments, operation A2 includes step (A2-1) and step (A2-2) described hereinafter. In step (A2-1), the initial product is washed with a washing solvent at a temperature ranging from about 40° C. to about 80° C. to remove the excess trialkyl phosphate, thereby obtaining a washed mixture. In some embodiments, the washing solvent may be ethyl acetate, butyl acetate, n-hexane, or the like, or a combination thereof. For example, the initial product obtained in operation A1 may be cooled to a temperature ranging from about 40° C. to about 80° C. A washing solvent, such as ethyl acetate, is then added to dissolve the trialkyl phosphate, and the washing act may be repeated multiple times. The washing act may be performed at a temperature of about 40° C., about 50° C., about 60° C., about 70° C., or about 80° C., for example.

In step (A2-2), the washing solvent is removed from the washed mixture to obtain the ionic liquid. In some embodiments, step (A2-2) includes evaporating the washing solvent to obtain the ionic liquid including the cation as shown in Formula (1) and the anion as shown in Formula (2). In some examples, the washing solvent may be evaporated by rotary evaporation under reduced pressure, vacuum evaporation, or a similar technique.

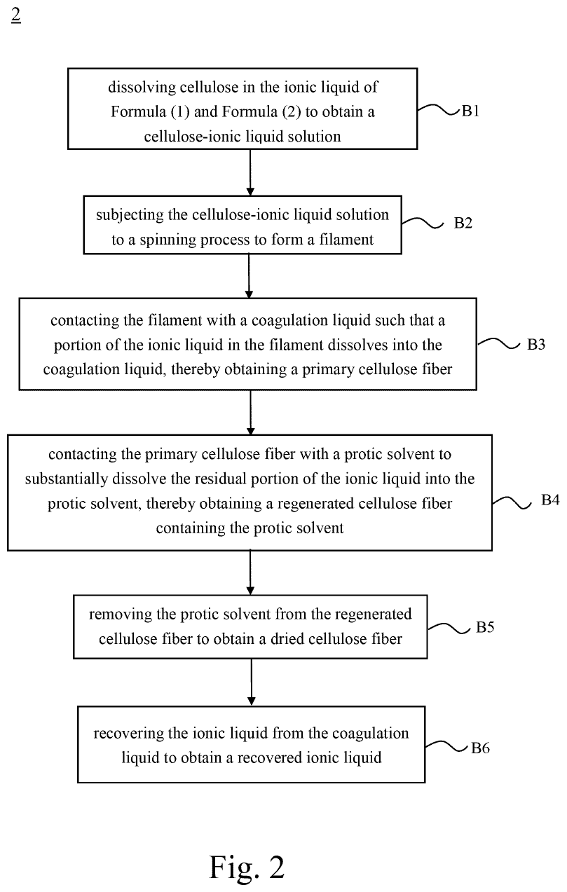

According to yet another aspect of the present disclosure, a method for spinning cellulose and recovering an ionic liquid is provided. FIG. 2 is a flowchart illustrating a method 2 for spinning cellulose and recovering an ionic liquid, according to some embodiments of the present disclosure. The method 2 includes operation B1, operation B2, operation B3, operation B4, operation B5, and operation B6.

In operation B1, cellulose is dissolved in the ionic liquid described hereinbefore in connection with Formula (1) and Formula (2) to obtain a cellulose-ionic liquid solution. In some embodiments, operation B1 is carried out at a temperature ranging from about 80° C. to about 120° C., for example, about 80° C., about 90° C., about 100° C., about 110° C., or about 120° C., depending on the specific type of ionic liquid used. In some embodiments, the cellulose includes wood pulp and/or non-wood plant pulp. Examples of wood pulp include paper grade pulp, dissolving pulp, fluff pulp, or any combination thereof. Examples of non-wood plant pulp include bamboo pulp, cotton pulp, other non-wood plant pulps, or any combination thereof.

In operation B2, the cellulose-ionic liquid solution is subjected to a spinning process to form a filament. Any suitable spinning technique known in the art may be used, for example, a dry-jet wet spinning process. In some embodiments, the cellulose-ionic liquid solution in step (B2) is spun at a temperature ranging from about 85° C. to about 115° C., for example, about 85° C., about 90° C., about 95° C., about 100° C., about 105° C., about 110° C., or about 115° C.

In operation B3, the filament is brought into contact with a coagulation liquid such that a portion of the ionic liquid in the filament diffuses and dissolves into the coagulation liquid, thereby obtaining a primary cellulose fiber. The primary cellulose fiber contains a residual portion of the ionic liquid. The coagulation liquid extracts a portion of the ionic liquid from the filament, allowing the filament to coagulate into the primary cellulose fiber while simultaneously cooling the filament. In some examples, the filament is introduced into a coagulation liquid at a temperature ranging from about 10° C. to about 30° C., for example, about 10° C., about 15° C., about 20° C., about 25° C., or about 30° C. In some embodiments, the coagulation liquid includes 0-30 wt % of the ionic liquid and 70-100 wt % of a protic solvent. Examples of the protic solvent include, but are not limited to, water, ethanol, propanol, or any combination thereof.

In operation B4, the primary cellulose fiber is brought into contact with a protic solvent such that the residual portion of the ionic liquid in the primary cellulose fibers substantially diffuses and dissolves into the protic solvent, thereby obtaining a regenerated cellulose fiber containing the protic solvent. The weight percentage of the ionic liquid remaining in the regenerated cellulose fiber containing the protic solvent is less than 0.1 wt %. In operation B4, examples of the protic solvent include, but are not limited to, water, ethanol, propanol, or mixtures thereof.

In operation B5, the protic solvent is removed from the regenerated cellulose fiber to obtain a dried cellulose fiber. In some embodiments, the regenerated cellulose fiber obtained from operation B4 is air-dried at room temperature to allow the protic solvent to evaporate, thereby obtaining the dried cellulose fiber. Any suitable technique known in the art may be used to remove the protic solvent from the fiber, for example, heating or reduced-pressure drying.

In operation B6, the ionic liquid is recovered from the coagulation liquid to obtain a recovered ionic liquid. As the ionic liquid described in operation B3 diffuses and dissolves into the coagulation liquid, an ionic liquid solution including the coagulation liquid and the dissolved ionic liquid is obtained. The protic solvent in the ionic liquid solution may be removed by any suitable technique known in the art, thereby obtaining the recovered ionic liquid. In some embodiments, the ionic liquid solution is concentrated by rotary evaporation under reduced pressure to obtain the recovered ionic liquid. In some embodiments, the recovered ionic liquid obtained in operation B6 is reused in operation B1, thereby being recycled in method 2.

It should be noted that, in conventional technologies, after an ionic liquid has been used to dissolve cellulose, its properties may deteriorate, resulting in a reduced ability of the recovered ionic liquid to dissolve cellulose upon reuse. However, according to embodiments of the present disclosure, for the embodiments in which Formula (1) and Formula (2) described hereinbefore have (i) R1 as an isopropyl group and R2 as a methyl group or an ethyl group, or (ii) R1 as a propyl group and R2 as an ethyl group, unexpected technical effects are achieved. The ionic liquids described in these embodiments exhibit substantially no deterioration in properties after being recycled 3 times, and maintain a cellulose dissolution capability substantially equivalent to that of the initial dissolution.

In some embodiments, as described above, the trialkyl phosphate in operation A1 is used in excess to ensure that the N-alkylimidazole is substantially completely reacted. N-alkylimidazole exhibits basicity upon contact with water, which may cause side reactions between cellulose and N-alkylimidazole. Such side reactions can reduce the dissolution capability of the recovered ionic liquid, decrease the spinnability of cellulose, impair the properties of the resulting fibers, and limit the usability of the recovered ionic liquid. Accordingly, in some embodiments of the present disclosure, the ionic liquid is substantially free of N-alkylimidazole, providing beneficial technical effects. Furthermore, in some embodiments, when the ionic liquid is diluted with water to form an aqueous solution having an ionic liquid concentration of about 10 wt % to about 90 wt %, the pH is maintained below 6, within an acidic range. The acidic environment minimizes the occurrence of side reactions after cellulose is dissolved in the ionic liquid.

Another aspect of the present disclosure provides a cellulose-ionic liquid solution including the ionic liquid described hereinbefore in connection with Formula (1) and Formula (2), and cellulose dissolved in the ionic liquid, in which the cellulose-ionic liquid solution is substantially free of N-alkylimidazole.

Yet another aspect of the present disclosure provides a method for dissolving cellulose. The method for dissolving cellulose includes operations of: (C1) dissolving cellulose in the ionic liquid described above in connection with Formula (1) and Formula (2) to obtain a cellulose-ionic liquid solution; (C2) separating the ionic liquid from the cellulose-ionic liquid solution to obtain a recovered ionic liquid; and (C3) dissolving cellulose, which is identical to or different from the cellulose in operation (C1), using the recovered ionic liquid. It is noted that, for an identical cellulose, the difference in solubility between the recovered ionic liquid in operation (C3) and the ionic liquid in operation (C1) is less than 0.5 wt %.

Examples 1-3 and Comparative Examples 1-13: Synthesis of Ionic Liquids, Preparation of Cellulose-Ionic Liquid Solutions, and Dissolution Temperature

N-alkylimidazole (1 mmol) was placed in a three-neck round-bottom flask and heated to 140° C. Under reflux conditions, a trialkyl phosphate (1.1 mmol) was added dropwise. Depending on the specific alkyl groups of the reactants, the reaction temperature ranged generally from about 140° C. to about 170° C., and the reaction time ranged from about 4 hours to about 12 hours. The reaction scheme is as shown in Formula (3) hereinbefore. During the reaction, thin-layer chromatography (TLC) was used to monitor the reaction to ensure that the N-alkylimidazole was substantially completely reacted. After completion of the reaction, the resultant mixture was cooled to about 60° C., and an equal weight of pure ethyl acetate was added for washing to remove excess trialkyl phosphate. This washing step was repeated three times, and TLC was used to confirm that no residual trialkyl phosphate remained. The resulting precipitate was collected in a single-neck round-bottom flask, and the washing solvent was removed using a rotary evaporator under reduced pressure to obtain a dialkylimidazolium dialkyl phosphate-type ionic liquid. In Examples 1-3 and Comparative Examples 1-13, the specific alkyl groups corresponding to Formula (3) are summarized in Table 1 below.

TABLE 1
Alkyl groups and cellulose dissolution temperature

| Example/Comparative | | | Dissolution |
|---|---|---|---|
| Example | R1 | R2 | Temperature (° C.) |
| Example 1 | isopropyl | methyl | 80 |
| Example 2 | isopropyl | ethyl | 110 |
| Example 3 | propyl | ethyl | 120 |
| Comparative Example 1 | methyl | methyl | >120 |
| Comparative Example 2 | methyl | ethyl | 80 |
| Comparative Example 3 | methyl | butyl | >120 |
| Comparative Example 4 | methyl | isobutyl | >120 |
| Comparative Example 5 | ethyl | methyl | 80 |
| Comparative Example 6 | ethyl | ethyl | 105 |
| Comparative Example 7 | ethyl | butyl | >120 |
| Comparative Example 8 | ethyl | isobutyl | >120 |
| Comparative Example 9 | propyl | methyl | 90 |
| Comparative Example 10 | propyl | butyl | >120 |
| Comparative Example 11 | propyl | isobutyl | >120 |
| Comparative Example 12 | isopropyl | butyl | >120 |
| Comparative Example 13 | isopropyl | isobutyl | >120 |

A mixture including cellulose and the ionic liquid at a cellulose content of 10 wt % was prepared and stirred at 10,000 rpm for 10 minutes. The cellulose was bleached softwood kraft pulp purchased from ARAUCO, Chile. The mixture was then heated to a temperature above 80° C. and maintained in a vacuum oven for 1 hour. Depending on the dissolution state of the cellulose, the heating temperature was adjusted to determine the temperature at which each ionic liquid was capable of dissolving cellulose (also referred to as the dissolution temperature). The results are summarized in Table 1 as well. When the temperature reached 120° C. and the ionic liquid was still unable to effectively dissolve the cellulose, the experiment was terminated, and the dissolution temperature was recorded as “>120” in Table 1.

### Example 4: Spinning Process of the Cellulose-Ionic Liquid Solution

Cellulose was dissolved using the ionic liquid of Example 1, and fiber spinning was carried out using a dry-jet wet spinning apparatus. The cellulose-ionic liquid solutions were fed into the spinning apparatus and extruded through a spinneret to form filaments. During the spinning process, the temperature of the cellulose-ionic liquid solution was maintained at 105° C. The filaments were then introduced into a 40-liter coagulation bath for coagulation, followed by washing in an 80-liter water bath containing a protic solvent to remove residual ionic liquid. The temperatures of both the coagulation bath and the washing water bath were maintained at 25° C. After washing, in a series of spinning experiments, the fibers were collected on godet rollers at respective draw ratios ranging from 1.2 to 3.1. The spinneret used was a single-hole nozzle having a capillary diameter of 150 μm. The washing water was replaced once per hour. Finally, the fibers were air-dried at room temperature for 48 hours to remove water, thereby obtaining dried fibers. The tensile strengths of the fibers were then measured.

The experimental results showed that when the draw ratio increased from 1.2 to 3.1, the tensile strength of the obtained fibers increased from 24.5 cN/tex to 36.90 cN/tex, which falls within the commonly reported range for NMMO systems (Jiang et al., 2020), namely from 35 cN/tex to 47 cN/tex. Meanwhile, the linear density (fineness) of the fibers decreased from 24.84 dtex to 8.63 dtex. Due to the limitation that the maximum draw ratio of the equipment used was only 3.1, the resultant fiber fineness reached only 8.63 dtex, which is significantly higher than the typical range of 1.3 dtex to 1.7 dtex for NMMO systems. These results indicate that, if the draw ratio can be further increased, the tensile strength of the fibers may be further improved.

### Example 5: Reuse of the Recycled Ionic Liquid

After completion of the spinning process of Example 4, an ionic liquid solution was obtained in the coagulation bath. Under reduced pressure, the protic solvent in the ionic liquid solution was evaporated using a rotary vacuum evaporator to obtain a recovered ionic liquid. Nuclear magnetic resonance (NMR) analysis of the recovered ionic liquid showed no detectable structural changes. The detailed NMR data are as follows:

¹H-NMR (600 MHz, CDCl₃-d) δ (ppm): 1.55 (d, J=6.72 Hz, 6H, NCHCH₃CH₃), 3.56 (s, 3H, P(OCH₃)₂), 3.58 (s, 3H, P(OCH₃)₂), 4.04 (s, 3H, NCH₃), 4.77 (m, 1H, NCHCH₃CH₃), 7.35 (t, J=1.8 Hz, 1H, NCHCHN), 7.43 (t, J=1.68 Hz, 1H, NCHCHN), 10.67 (s, 1H, NCHN). ¹³C-NMR (150 MHz, CDCl₃-d) δ (ppm): 22.99, 36.26, 52.41, 52.45, 52.95, 119.26, 123.32, 138.56.

The recovered ionic liquid obtained from Example 4 was reused to dissolve cellulose, followed by spinning and subsequent recovery of the ionic liquid according to the same procedures described above. This cycle was repeated three times to evaluate the reusability of the ionic liquid. In each cycle, the recycled ionic liquid was analyzed by NMR spectroscopy to confirm structural integrity thereof, and the tensile strengths of the fibers and their fineness values were measured. The results are summarized in Table 2 below.

TABLE 2
Fiber properties obtained using recycled ionic liquids

No. of Recycling
Cycles

| | | 1^{st} | 2^{nd} | 3^{rd} |
|---|---|---|---|---|
| Fiber Properties | Initial | Recycle | Recycle | Recycle |
| Fineness (dtex) | 8.63 | 8.30 | 8.37 | 8.52 |
| Tensile strength | 36.90 | 36.98 | 37.08 | 36.92 |
| (cN/tex) | | | | |

The results in Table 2 demonstrate that, after three cycles of cellulose dissolution and spinning, the recycled ionic liquid still retained its ability to dissolve cellulose, and the tensile strength of the regenerated fibers remained substantially unchanged. Similar results were also obtained when the ionic liquids of Example 2 and Example 3 were used. These results confirm the structural stability and recyclability of the ionic liquids and highlight the potential of the ionic liquids for application in sustainable cellulose fiber production.

In contrast, when the ionic liquids of Comparative Example 2, Comparative Example 5, Comparative Example 6, or Comparative Example 9 shown in Table 1 were used in experiments similar to those described in Example 4 and Example 5 (with only the type of ionic liquid being changed), the recycled ionic liquids exhibited a considerable decrease in their ability to re-dissolve cellulose after a single cycle of cellulose dissolution and spinning.

According to some embodiments of the present disclosure, an ionic liquid is provided. The ionic liquid includes a cation and an anion. The cation has a structure represented by Formula (1),

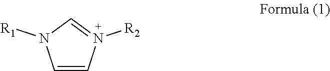

and the anion has a structure represented by Formula (2),

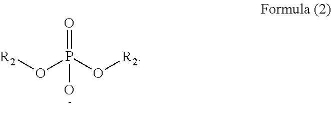

In Formula (1) and Formula (2), R1 and R2 are defined as follows: (i) R1 is an isopropyl group and R2 is a methyl group or an ethyl group; or (ii) R1 is a propyl group and R2 is an ethyl group.

According to yet some embodiments of the present disclosure, a method for producing an ionic liquid is provided. The method includes operations of: (A1) reacting an N-alkylimidazole with a trialkyl phosphate, wherein the trialkyl phosphate is present in excess and the N-alkylimidazole is substantially completely reacted, to obtain an initial product; and (A2) removing the excess trialkyl phosphate from the initial product to obtain the ionic liquid described above in connection with Formula (1) and Formula (2).

In some embodiments, in operation (A1), a molar ratio of the trialkyl phosphate to the N-alkylimidazole is from about 1.05 to about 1.15.

In some embodiments, operation (A1) includes reacting the N-alkylimidazole with the trialkyl phosphate at a temperature ranging from about 130° C. to about 170° C. for a time period of about 3 hours to about 8 hours, to obtain the initial product.

In some embodiments, operation (A2) includes steps of: (A2-1) washing the initial product with a washing solvent at a temperature ranging from about 40° C. to about 80° C. to remove the excess trialkyl phosphate and obtain a washed mixture; and (A2-2) removing the washing solvent from the washed mixture to obtain the ionic liquid.

In some embodiments, the washing solvent includes ethyl acetate, butyl acetate, n-hexane, or a combination thereof.

In some embodiments, step (A2-2) includes evaporating the washing solvent to obtain the ionic liquid.

According to yet some embodiments of the present disclosure, a method for spinning cellulose and recycling an ionic liquid is provided. The method includes operations of: (B1) dissolving cellulose in the ionic liquid of claim 1 to obtain a cellulose-ionic liquid solution; (B2) subjecting the cellulose-ionic liquid solution to a spinning process to form a filament; (B3) contacting the filament with a coagulation liquid such that a portion of the ionic liquid in the filament dissolves into the coagulation liquid, thereby obtaining a primary cellulose fiber having a residual portion of the ionic liquid; (B4) contacting the primary cellulose fiber with a protic solvent to substantially dissolve the residual portion of the ionic liquid into the protic solvent, thereby obtaining a regenerated cellulose fiber containing the protic solvent; (B5) removing the protic solvent from the regenerated cellulose fiber to obtain a dried cellulose fiber; and (B6) recovering the ionic liquid from the coagulation liquid to obtain a recovered ionic liquid.

In some embodiments, in operation (B2), the spinning process is performed at a temperature that ranges from about 85° C. to about 115° C.

In some embodiments, the method further includes reusing the recovered ionic liquid obtained in operation (B6) in operation (B1).

In some embodiments, the cellulose includes wood pulp and/or non-wood plant pulp, wherein the wood pulp includes paper grade pulp, dissolving pulp, fluff pulp, or any combination thereof, and wherein the non-wood plant pulp includes bamboo pulp, cotton pulp, or a combination thereof.

According to yet some embodiments of the present disclosure, a method for dissolving cellulose is provided. The method includes operations of: (C1) dissolving cellulose in the ionic liquid of claim 1 to obtain a cellulose-ionic liquid solution, wherein the cellulose-ionic liquid solution is substantially free of N-alkylimidazole; (C2) separating the ionic liquid from the cellulose-ionic liquid solution to obtain a recovered ionic liquid; and (C3) dissolving cellulose, which is identical to or different from the cellulose in operation (C1), using the recovered ionic liquid, wherein a difference in solubility between the recovered ionic liquid in operation (C3) and the ionic liquid in operation (C1) is less than 0.5 wt % for an identical cellulose.

According to yet some embodiments of the present disclosure, a cellulose-ionic liquid solution is provided. The cellulose-ionic liquid solution includes the ionic liquid described above in connection with Formula (1) and Formula (2) and a cellulose dissolved in the ionic liquid, wherein the cellulose-ionic liquid solution is substantially free of N-alkylimidazole.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. An ionic liquid, comprising a cation and an anion,
wherein the cation has a structure represented by Formula (1): 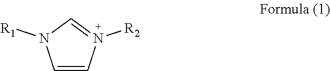
and wherein the anion has a structure represented by Formula (2): 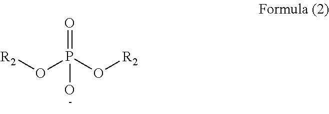
wherein
(i) R1 is an isopropyl group and R2 is a methyl group or an ethyl group; or
(ii) R1 is a propyl group and R2 is an ethyl group.

2. A method for producing an ionic liquid, comprising operations of:
(A1) reacting an N-alkylimidazole with a trialkyl phosphate, wherein the trialkyl phosphate is present in excess and the N-alkylimidazole is substantially completely reacted, to obtain an initial product; and
(A2) removing the excess trialkyl phosphate from the initial product to obtain the ionic liquid of claim 1.

3. The method of claim 2, wherein, in operation (A1), a molar ratio of the trialkyl phosphate to the N-alkylimidazole is from about 1.05 to about 1.15.

4. The method of claim 2, wherein operation (A1) comprises reacting the N-alkylimidazole with the trialkyl phosphate at a temperature ranging from about 130° C. to about 170° C. for a time period of about 3 hours to about 8 hours, to obtain the initial product.

5. The method of claim 2, wherein operation (A2) comprises steps of:
(A2-1) washing the initial product with a washing solvent at a temperature ranging from about 40° C. to about 80° C. to remove the excess trialkyl phosphate and obtain a washed mixture; and
(A2-2) removing the washing solvent from the washed mixture to obtain the ionic liquid.

6. The method of claim 5, wherein the washing solvent comprises ethyl acetate, butyl acetate, n-hexane, or a combination thereof.

7. The method of claim 5, wherein step (A2-2) comprises evaporating the washing solvent to obtain the ionic liquid.

8. A method for spinning cellulose and recycling an ionic liquid, comprising operations of:
(B1) dissolving cellulose in the ionic liquid of claim 1 to obtain a cellulose-ionic liquid solution;
(B2) subjecting the cellulose-ionic liquid solution to a spinning process to form a filament;
(B3) contacting the filament with a coagulation liquid such that a portion of the ionic liquid in the filament dissolves into the coagulation liquid, thereby obtaining a primary cellulose fiber having a residual portion of the ionic liquid;
(B4) contacting the primary cellulose fiber with a protic solvent to substantially dissolve the residual portion of the ionic liquid into the protic solvent, thereby obtaining a regenerated cellulose fiber containing the protic solvent;
(B5) removing the protic solvent from the regenerated cellulose fiber to obtain a dried cellulose fiber; and
(B6) recovering the ionic liquid from the coagulation liquid to obtain a recovered ionic liquid.

9. The method of claim 8, wherein, in operation (B2), the spinning process is performed at a temperature that ranges from about 85° C. to about 115° C.

10. The method of claim 8, further comprising reusing the recovered ionic liquid obtained in operation (B6) in operation (B1).

11. The method of claim 8, wherein the cellulose comprises wood pulp and/or non-wood plant pulp, wherein the wood pulp comprises paper grade pulp, dissolving pulp, fluff pulp, or any combination thereof, and wherein the non-wood plant pulp comprises bamboo pulp, cotton pulp, or a combination thereof.

12. A method for dissolving cellulose, comprising operations of:
(C1) dissolving cellulose in the ionic liquid of claim 1 to obtain a cellulose-ionic liquid solution, wherein the cellulose-ionic liquid solution is substantially free of N-alkylimidazole;
(C2) separating the ionic liquid from the cellulose-ionic liquid solution to obtain a recovered ionic liquid; and
(C3) dissolving cellulose, which is identical to or different from the cellulose in operation (C1), using the recovered ionic liquid;
wherein a difference in solubility between the recovered ionic liquid in operation (C3) and the ionic liquid in operation (C1) is less than 0.5 wt % for an identical cellulose.

13. A cellulose-ionic liquid solution, comprising:
the ionic liquid of claim 1; and
a cellulose dissolved in the ionic liquid;
wherein the cellulose-ionic liquid solution is substantially free of N-alkylimidazole.
